SEQUENCE LISTING (2 sequences)

SEQ ID NO: 1 - DNA, 2047 bp, synthetic construct
  source 1..2047: mol_type = other DNA
atgagcatctcggtgaacgggcagtcgtgcgtgccgccggggttccgcttccaccccacggaggaggagctgctcaactactacctccgcaagaaggtggcctcccaggagatcgacctcgacgtcatccgcgacgtcgacctcaacaagctcgagccatgggacatccaaggtacgtacgtacgcacggccggtccggtccgatccggtaccactgccttcttcaacttcaagttcaagcacgcgcgcgcgcagtagcagagcagctcgtggtcgtggatcagatcggatcggagctggtgcacgaccatgagagcacggacatgaacatgaacagaccttgttgtgaatgcaactgccttagctagctaccaagcaagtactctcgttcgtacagcagcatgtatagcttatgttcgttgatcgacaaaactagctagcaaacaatcaaacgcgatcgaattcatcgcgcgctaactactggctaacaactgctactactactaaagccgctagctccattccatgcatggaaatcgcgcgcagagaaatgcaagatcgggtcgggtccccagaacgactggtacttcttcagccacaaggacaagaagtacccgacggggacgcgcaccaaccgcgccacggccgccgggttctagaaggccaccggccgcgacaaggccatctacaacgccgtcaagcgcatcggcatgcgcaagacgctcgtcttctacaagggccgcgcgccgcacggccagaagtccgactggatcatgcacgagtaccgcctcgacgaccccgctgctgctgctgctgctggatccggtgatgccgtggccaacgacgacgcagccgccacggtaagcaaagcaacgaccctgatcgccgttaatctcttctctgcaccaccagttcacgtacgccaccattaataattgcctgccgtaagataagaaacaattatatggcggtggtggtgcaatcatgcgagtacggcgacccgccttgatttgatccagctccaggctcaaggctccggccgtatttttttccgctctcttgttttgattgattgatgaggaggagagagagagcagtaggcgctagctactagctagctaggggaaggagggagggacggacgtagtaataattattaaactttgccatgtgcctcatgtgccccaaaaggtagcaataattaacactgctgcactgtttttttttaatctgcttcttgtcgacttgtcgtcggcggcgatgtcgcggtggacaggctgctgctgctgccgccgcgtcgtcggacggcgggcaggaggacggctgggtggtgtgcagggtgttcaagaagaagcaccaccacaaggagtcaggtgggggcgggggcaacaagcacggcagcagtaacagcgagcatgggcacggcggcgccggcaaggcatcggctgcggctgcggctgcggcgcaccagcaccagcaccatggaggcctgcagtactcctccagcgacgaggcgctggaccagatcctgcagtacatgggcaggtcgtgcaagcaggagcacgagctggtgtcgccggcgccggcgccgccgggacgggcggcggcgtccaggtacctccggcccatcgagaccgttctgggcgggcacgcgttcatgaagcttcccgcgctcgagagcccgtccgcggccgcgtccgcatcgctgacacagccggcgcagcacgacgagctctaccgcgccgccgggaacgggatcacggactgggccatgatggaccggctggtggcgtcgcacctgaacgggcagcaggcgcccgccgcggcggaccagctcggcggcggctgcggcttcgacgcggacgccggcgccgaagacgcggacgccggcctcgccttctactccgccgccgccagccggctgctcggctccggcggcggcgccggcagcgacgacgacctgtggagcttcacgcggtcgtcggtttcgtcaacggcggcggcggcggccacgtccacggagcggctcagccacgtgtcactgtag

SEQ ID NO: 2 - DNA, 2047 bp, synthetic construct
  source 1..2047: mol_type = other DNA
atgagcatctcggtgaacgggcagtcgtgcgtgccgccggggttccgcttccaccccacggaggaggagctgctcaactactacctccgcaagaaggtggcctcccaggagatcgacctcgacgtcatccgcgacgtcgacctcaacaagctcgagccatgggacatccaaggtacgtacgtacgcacggccggtccggtccgatccggtaccactgccttcttcaacttcaagttcaagcacgcgcgcgcgcagtagcagagcagctcgtggtcgtggatcagatcggatcggagctggtgcacgaccatgagagcacggacatgaacatgaacagaccttgttgtgaatgcaactgccttagctagctaccaagcaagtactctcgttcgtacagcagcatgtatagcttatgttcgttgatcgacaaaactagctagcaaacaatcaaacgcgatcgaattcatcgcgcgctaactactggctaacaactgctactactactaaagccgctagctccattccatgcatggaaatcgcgcgcagagaaatgcaagatcgggtcgggtccccagaacgactggtacttcttcagccacaaggacaagaagtacccgacggggacgcgcaccaaccgcgccacggccgccgggttctggaaggccaccggccgcgacaaggccatctacaacgccgtcaagcgcatcggcatgcgcaagacgctcgtcttctacaagggccgcgcgccgcacggccagaagtccgactagatcatgcacgagtaccgcctcgacgaccccgctgctgctgctgctgctggatccggtgatgccgtggccaacgacgacgcagccgccacggtaagcaaagcaacgaccctgatcgccgttaatctcttctctgcaccaccagttcacgtacgccaccattaataattgcctgccgtaagataagaaacaattatatggcggtggtggtgcaatcatgcgagtacggcgacccgccttgatttgatccagctccaggctcaaggctccggccgtatttttttccgctctcttgttttgattgattgatgaggaggagagagagagcagtaggcgctagctactagctagctaggggaaggagggagggacggacgtagtaataattattaaactttgccatgtgcctcatgtgccccaaaaggtagcaataattaacactgctgcactgtttttttttaatctgcttcttgtcgacttgtcgtcggcggcgatgtcgcggtggacaggctgctgctgctgccgccgcgtcgtcggacggcgggcaggaggacggctgggtggtgtgcagggtgttcaagaagaagcaccaccacaaggagtcaggtgggggcgggggcaacaagcacggcagcagtaacagcgagcatgggcacggcggcgccggcaaggcatcggctgcggctgcggctgcggcgcaccagcaccagcaccatggaggcctgcagtactcctccagcgacgaggcgctggaccagatcctgcagtacatgggcaggtcgtgcaagcaggagcacgagctggtgtcgccggcgccggcgccgccgggacgggcggcggcgtccaggtacctccggcccatcgagaccgttctgggcgggcacgcgttcatgaagcttcccgcgctcgagagcccgtccgcggccgcgtccgcatcgctgacacagccggcgcagcacgacgagctctaccgcgccgccgggaacgggatcacggactgggccatgatggaccggctggtggcgtcgcacctgaacgggcagcaggcgcccgccgcggcggaccagctcggcggcggctgcggcttcgacgcggacgccggcgccgaagacgcggacgccggcctcgccttctactccgccgccgccagccggctgctcggctccggcggcggcgccggcagcgacgacgacctgtggagcttcacgcggtcgtcggtttcgtcaacggcggcggcggcggccacgtccacggagcggctcagccacgtgtcactgtag

(19) United States
(12) Patent Application Publication
(10) Pub. No.: US 2025/0230461 A1
(43) Pub. Date: Jul. 17, 2025

(54) MUTANT GENE OF ZMNST2 AND METHOD FOR IMPROVING EFFICIENCY OF BIOETHANOL PRODUCTION THROUGH FERMENTATION OF MAIZE STRAW

(71) Applicant: Henan Agricultural University, Zhengzhou (CN)

(72) Inventors: Mingyue GOU, Zhengzhou (CN); Ying WANG, Zhengzhou (CN); Yanwen YU, Zhengzhou (CN); Jiankun LI, Zhengzhou (CN); Ye XING, Zhengzhou (CN); Chenyang ZHAO, Zhengzhou (CN); Mengyu YUAN, Zhengzhou (CN)

(21) Appl. No.: 19/027,618

(22) Filed: Jan. 17, 2025

Related U.S. Application Data
(63) Continuation of application No. PCT/CN2024/111052, filed on Aug. 9, 2024.

(30) Foreign Application Priority Data
Jan. 15, 2024 (CN) 202410052363.3

(51) Int. Cl. C12N 15/82

(52) U.S. Cl. CPC C12N 15/8255

(57) ABSTRACT

A mutant gene of ZmNST2 and a method for improving the efficiency of bioethanol production through fermentation of maize straw are provided. According to the disclosure, the mutant gene of ZmNST2 is obtained by molecular genetic methods and the effects of ZmNST2 mutation on lignin content, fermentation inhibitor content, ethanol yield, and hydrolysis rates of cellulase in maize are determined.

### CROSS-REFERENCE TO RELATED APPLICATIONS

This application is a continuation of PCT/CN2024/111052, filed on Aug. 9, 2024 and claims priority of Chinese Patent Application No. 202410052363.3, filed on Jan. 15, 2024, the entire contents of which are incorporated herein by reference.

### INCORPORATION BY REFERENCE STATEMENT

This statement, made under Rules 77 (b) (5) (ii) and any other applicable rule incorporates into the present specification of an XML file for a “Sequence Listing XML” (see Rule 831 (a)), submitted via the USPTO patent electronic filing system or on one or more read-only optical discs (see Rule 1.52 (e) (8)), identifying the names of each file, the date of creation of each file, and the size of each file in bytes as follows:
File name: PCT-US-2024-18161 Sequence Listing
Creation date: 16 Jan. 2025
Byte size: 6,832

### TECHNICAL FIELD

The present disclosure relates to genetic engineering breeding, and in particular to a mutant gene of ZmNST2 and a method for improving the efficiency of bioethanol production through fermentation of maize straw.

### BACKGROUND

Energy is a necessity for human survival. Fossil fuels, as the primary energy source, have made tremendous contributions to the development of human society. At present, the utilization of fossil fuels not only has adverse effects on the environment, such as air pollution and soil pollution, but also is becoming increasingly scarce, thus posing a huge threat to human survival. Now, it is urgent to find a sustainable and green energy source. Bioethanol, with its green and renewable characteristics, has become a hot spot in the field of energy research. First generation bioethanol mainly uses sugar and starch from grain as fermentation raw materials. It is initially to solve the problem of hoarding of old grain, but with the rapid development of bioethanol based on grain as raw material, a large amount of old grain is consumed, and then the new grain began to be consumed. It not only has an impact on the food security of China, but also does not conform to the concept of sustainable development. Second-generation bioethanol is produced from lignocellulosic biomass. The lignocellulosic biomass is one of the richest natural renewable carbohydrate resources, and one of its raw materials is crop straw. Therefore, lignocellulosic biomass has a very promising future in industrial production due to the advantages of not competing with people for grain, stable yield, and alleviation of atmospheric and environmental pollution problems caused by the disposal and burning of straw in the traditional production process.

The production of cellulosic ethanol from lignocellulose is mainly based on cellulose and hemicellulose produced after pretreatment of lignocellulose. The cellulose and hemicellulose produce fermentable sugars (mainly glucose and xylose) after enzymatic hydrolysis, and then the fermentable sugars are fermented to produce ethanol. Lignocellulose is mainly composed of cellulose, hemicellulose and lignin, and its structure is made up of cellulose tightly arranged inside, lignin wrapped around the outside, and hemicellulose connected in the middle. The wrap of lignin has a supportive and protective effect on the surface of lignocellulosic fibers, but it is difficult for cellulase used for hydrolysis to hydrolyze the internal cellulose and hemicellulose because of their high hydrophobicity, polymerization, and chemical stability. Dilute acid hydrolysis is the most commonly used chemical pretreatment method for enzymatic hydrolysis of lignocellulose. Through chemical pretreatment, most of the hemicellulose can be dissolved and the structure of lignin can be disrupted to increase the rate of hydrolysis of cellulose, and thereby increases the amount of ethanol produced by fermentation. However, during pretreatment, soluble inhibitors such as furfural and phenolic compounds are generated, these usually inhibit fermentation and further reduce ethanol production. Maize is one of the widely grown crops in China, and has a high potential for lignocellulosic ethanol generation because it has a higher stover biomass than crops such as wheat and rice. Mining the genes affecting ethanol production from maize straw fermentation is of great significance for breeding new maize varieties with high ethanol yield by fermentation, bioethanol production, energy sustainability and environmental improvement.

### SUMMARY

An objective of the present disclosure is to provide a mutant gene of ZmNST2 and a method for improving the efficiency of bioethanol production through fermentation of maize straw, so as to solve the problems existing in the prior art. In this disclosure, a mutant gene of ZmNST2 is obtained, which may reduce lignin content, reduce fermentation inhibitors and improve the hydrolysis rate of cellulase and the yield of fermented ethanol. The mutant gene plays a key role in the application of maize stalk to produce bioethanol and is an important resource for producing bioethanol from maize stalk.

In order to achieve the above objective, the present disclosure provides the following scheme.

The present disclosure provides a mutant gene of ZmNST2, and a nucleotide sequence of the mutant gene is shown in SEQ ID NO: 1 or SEQ ID NO: 2.

The present disclosure provides a biomaterial including the above-mentioned mutant gene of ZmNST2, and the biomaterial includes an expression vector or a recombinant bacterium.

The present disclosure provides a method for creating a maize mutant, and the method includes introducing the above-mentioned mutant gene of ZmNST2 or the above-mentioned biomaterial into a maize material to obtain the maize mutant.

The present disclosure provides a method for improving efficiency of bioethanol production through fermentation of maize straw, and the method includes the steps of producing the bioethanol by fermentation of the maize mutant.

Further, the efficiency of the bioethanol production by fermentation is improved by reducing a lignin content in the maize straw.

Further, the efficiency of the bioethanol production by fermentation is improved by reducing a content of fermentation inhibitor of the maize straw.

Further, the efficiency of the bioethanol production by fermentation is improved by increasing a hydrolysis rate of cellulase in the maize straw.

The disclosure has the following effects.

The present disclosure finds that the zmnst2 mutant plant has a phenotype of distinct leaf softening compared to wild type maize plants. Through determination results of lignin level and ethanol fermentation level, the zmnst2 mutant plant is found to have reduced lignin level, reduced level of fermentation inhibitors, and increased hydrolysis rate of cellulase and fermentation level for ethanol production compared to the wild type plant. Moreover, the present disclosure provides the mutant gene of ZmNST2 by molecular genetics, and it is found that the mutant gene is capable of reducing the lignin level in maize straw, reducing fermentation inhibitors and increasing the hydrolysis rate of cellulase after pretreatment of maize straw, and thereby improves the quality and efficiency of the fermentation to produce bioethanol. Thus, the mutant gene of ZmNST2 provided by the present disclosure has important application value for breeding new maize varieties with high ethanol yield by fermentation, bioethanol production, energy sustainability and environmental improvement.

### BRIEF DESCRIPTION OF THE DRAWINGS

For a clearer illustration of the technical schemes in the embodiments of the present disclosure or in the prior art, a brief description of the drawings to be used in the embodiments is given below. It is obvious that the drawings in the following description are only some embodiments of the present application and that other drawings are also available to those of ordinary skill in the art without any creative effort.

FIG. 1 shows the gene structure of ZmNST2, where zmnst2-1 G-A 658 bp and zmnst2-2 G-A 769 bp show physical locations of mutation sites of the zmnst2-1 mutant and zmnst2-2 mutant.

FIG. 2 shows lignin monomer content in roots of wild type B73 plant, zmnst2-1 mutant plant, and zmnst2-2 mutant plant, where different letters denote significant differences (P<0.05).

FIG. 3 shows lignin monomer content in internodes of wild type B73 plant, zmnst2-1 mutant plant, and zmnst2-2 mutant plant, where different letters denote significant differences (P<0.05).

FIG. 4 shows lignin monomer content in leaf veins of wild type B73 plant, zmnst2-1 mutant plant and zmnst2-2 mutant plant, where different letters denote significant differences (P<0.05).

FIG. 5 shows cellulose content in roots, internodes and leaf veins of wild type B73 plant, zmnst2-1 mutant plant, and zmnst2-2 mutant plant, where different letters denote significant differences (P<0.05).

FIG. 6 shows content of acid-insoluble lignin, glucan and xylan in wild-type B73 straw and zmnst2-1 mutant straw, where *P<0.05; **P<0.005; ***P<0.001, ns indicates insignificant difference.

FIG. 7 shows content of glucose and xylose in wild-type B73 straw and zmnst2-1 mutant straw after pretreatment by dilute acid, where *P<0.05; **P<0.005; ***P<0.001, ns indicates insignificant difference.

FIG. 8 shows content of fermentation inhibitor phenolic compounds (AT₂₈₀) in wild-type B73 straw and zmnst2-1 mutant straw after pretreatment by dilute acid, where *P<0.05; **P<0.005; ***P<0.001, ns indicates insignificant difference.

FIG. 9 shows content of toxicant furfural and 5-hydroxymethylfurfural in wild-type B73 straw and zmnst2-1 mutant straw after pretreatment by dilute acid, where *P<0.05; **P<0.005; ***P<0.001, ns indicates insignificant difference.

FIG. 10 shows content of ethanol 1 produced by wild-type B73 straw and zmnst2-1 mutant straw by co-fermentation of glucose and xylose and content of ethanol 2 produced by wild-type B73 straw and zmnst2-1 mutant straw by glucose-only fermentation after hydrolysis of cellulose by cellulase, where *P<0.05; **P<0.005; ***P<0.001, ns indicates insignificant difference.

FIG. 11 shows hydrolysis rates of cellulase in wild-type B73 straw and zmnst2-1 mutant straw, where *P<0.05; **P<0.005; ***P<0.001, ns indicates insignificant difference.

### DETAILED DESCRIPTION OF THE EMBODIMENTS

Various exemplary embodiments of the present disclosure are now described in detail, which should not be considered a limitation of the present disclosure, but rather should be understood as a more detailed description of certain aspects, features, and embodiments of the present disclosure.

It should be understood that the terms described in the present disclosure are only intended to describe particular embodiments and are not intended to limit the present disclosure. Further, for the range of values in the present disclosure, it should be understood that each intermediate value between the upper and lower limits of the range is also specifically disclosed. Each smaller range between any stated value or intermediate value within the stated range, and any other stated value or intermediate value within the stated range, is also included in this disclosure. The upper and lower limits of these smaller ranges may be independently included or excluded from the scope.

Unless otherwise indicated, all technical and scientific terms used herein have the same meanings as are commonly understood by those of ordinary skill in the art described in this disclosure. While this disclosure describes only various methods and materials, any methods and materials similar or equivalent to those described herein may also be used in the implementation or testing of this disclosure. All literature referred to in this specification is incorporated by reference to disclose and describe the methods and/or materials associated with said literature. In the event of a conflict with any incorporated literature, the content of this specification shall prevail.

Various improvements and variations may be made to specific embodiments of the specification of the present disclosure without departing from the scope or spirit of the present disclosure, as will be apparent to those skilled in the art. Other embodiments obtained from the specification of the present disclosure will be apparent to those skilled in the art. The present disclosure specification and embodiments are exemplary only.

As used herein, the terms “comprising,” “including,” “having,” “containing,” and the like are all open-ended terms, i.e., meant to include but not be limited to.

### Embodiment 1

### I. Experimental Materials and Experimental Method

### 1. Source of Materials

zmnst2-1 mutant and zmnst2-2 mutant are purchased from the Mutant Library in Maize (https://elabcaas.cn/memd/public/index.html#/, the original numbers of the mutants are: EMS4-134f89 and EMS4-1ad22e, respectively). Prior to this embodiment, no functional studies have been reported on the zmnst2 mutant, and in this embodiment, the zmnst2 mutant is studied for the first time and the mutant gene of ZmNST2 is obtained by molecular genetics. The details are shown below.

### 2. Experimental Method

### 2.1 Staining of Lignin

During field planting, the zmnst2 mutant is found to have a significant phenotype of leaf softening compared to wild type B73, and it is hypothesized that its lignin level may be reduced. Roots, internodes, and leaf veins of wild type B73, zmnst2-1 mutant, zmnst2-2 mutant, and zmnst2-1/zmnst2-2 (F1 generation obtained by crossing zmnst2-1 plant and zmnst2-2 plant) mutant are stained using phloroglucinol-hydrochloric acid staining (Wiesner), and then total lignin is observed. Specifically, it includes the following steps: firstly, the tissue specimens are cut into uniform slices of about 1 mm with a blade, and the slices are placed in 75% alcohol for dechlorophyllisation for later use; after that, the slices are placed in Wiesner staining solution until the color is developed; finally, the slices are placed in the body microscope for observation and photographed for deposit.

### 2.2 Determination of Lignin Level and Cellulose Level

### 2.2.1 Extraction of Cell Walls

Specifically, it includes the following steps: firstly, air-drying the samples and grinding it into powder, taking 70 mg of the powder, adding 1.5 mL of 70% ethanol into the 70 mg of the powder and then heating in 70° C. water bath and discarding the supernatant; adding 1.5 mL chloroform:methanol (Volume/Volume (v/v)=1:1), and then oscillating and resuspending; adding 1 mL of acetone, oscillating and resuspending, then discarding supernatant, where this process is repeated several times until the supernatant is colorless; drying the treated samples at 35° C. to obtain the cell walls.

### 2.2.2 Determination of Lignin Levels

Specifically, it includes the following steps: weighing samples of cell walls of different tissues (around 20 mg of root tissue and leaf tissue, and around 10 mg of internode tissue) separately, and determining the lignin levels of the samples by Gas Chromatography-Mass Spectrometer (GC-MS) using thiolysis.

### 2.2.3 Determination of Cellulose Levels

Specifically, it includes the following steps: weighing about 5 mg of samples of cell walls and treating them with 1 mL of acetic acid-nitric acid solution (acetic acid:nitric acid:water=8:1:2, v/v) to hydrolyzing cellulose to glucose, and then determining the glucose levels (which represent the cellulose levels) using anthrone colorimetry.

### 2.3 Measurement of Fermentation Indexes

Specifically, it includes the following steps: air-drying, pulverizing and sieving the maize straw to obtain maize straw powder, selecting 20-40 mesh maize straw powder, and determining the levels of acid-insoluble lignin, hemicellulose (glucose) and cellulose (xylan) by using National Renewable Energy Method for quantifying cellulose, hemicellulose and lignin (NREL method); pretreating the straw powder using dilute sulfuric acid hydrolysis to obtain hydrolysate, and determining the level of phenolic compounds (AT₂₈₀) in the hydrolysate using ultraviolet-visible spectrophotometry, and determining the levels of furfural, 5-hydroxymethylfurfural, glucose, and xylose in the hydrolysate using high-performance liquid chromatography (HPLC); fermenting detoxified and decolorized hydrolyzed sugar solution using Pichia stipitis (CICC1960, purchased from the China Center of Industrial Culture Collection) to produce ethanol (ethanol 1), and then determining the concentration of ethanol by HPLC; neutralizing the cellulose residue after pretreatment using NaHCO₃ firstly, then rinsing it with tap water, and finally hydrolyzing the dried cellulose using cellulase; fermenting the hydrolyzed sugar solution using Saccharomyces cerevisiae P1 (screened and deposited by Zhang Qin research group at Anhui Polytechnic University) to produce ethanol (ethanol 2), and then determining the concentration of ethanol by HPLC.

### II. Experimental Results

### 1. Phenotype of Zmnst2 Mutant

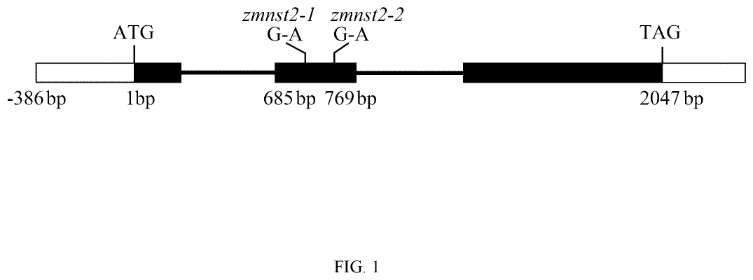

ZmNST2 has three exons, as shown in FIG. 1, zmnst2-1 mutant and zmnst2-2 mutant are on the second exon of the ZmNST2 gene respectively, single bases G to A mutated on the positions away from the start codon 658 bp and 769 bp, and thereby the translation of amino acids is terminated prematurely. Within this interval, other mutations causing loss of function of the gene could result in lower lignin level, reduced fermentation inhibitors and higher cellulase hydrolysis rate, so that the efficiency of bioethanol production by fermentation of maize straw may be improved.

The nucleotide sequences of the ZmNST2 mutant genes of zmnst2-1 mutant and zmnst2-2 mutant are shown in SEQ ID NO: 1-2:

zmnst2-1 mutant (SEQ ID NO: 1):
ATGAGCATCTCGGTGAACGGGCAGTCGTGCGTGCCGCCGGGGTTC
CGCTTCCACCCCACGGAGGAGGAGCTGCTCAACTACTACCTCCGC
AAGAAGGTGGCCTCCCAGGAGATCGACCTCGACGTCATCCGCGAC
GTCGACCTCAACAAGCTCGAGCCATGGGACATCCAAGGTACGTAC
GTACGCACGGCCGGTCCGGTCCGATCCGGTACCACTGCCTTCTTC
AACTTCAAGTTCAAGCACGCGCGCGCGCAGTAGCAGAGCAGCTCG
TGGTCGTGGATCAGATCGGATCGGAGCTGGTGCACGACCATGAGA
GCACGGACATGAACATGAACAGACCTTGTTGTGAATGCAACTGCC
TTAGCTAGCTACCAAGCAAGTACTCTCGTTCGTACAGCAGCATGT
ATAGCTTATGTTCGTTGATCGACAAAACTAGCTAGCAAACAATCA
AACGCGATCGAATTCATCGCGCGCTAACTACTGGCTAACAACTGC
TACTACTACTAAAGCCGCTAGCTCCATTCCATGCATGGAAATCGC
GCGCAGAGAAATGCAAGATCGGGTCGGGTCCCCAGAACGACTGGT
ACTTCTTCAGCCACAAGGACAAGAAGTACCCGACGGGGACGCGCA
CCAACCGCGCCACGGCCGCCGGGTTCTAGAAGGCCACCGGCCGCG
ACAAGGCCATCTACAACGCCGTCAAGCGCATCGGCATGCGCAAGA
CGCTCGTCTTCTACAAGGGCCGCGCGCCGCACGGCCAGAAGTCCG
ACTGGATCATGCACGAGTACCGCCTCGACGACCCCGCTGCTGCTG
CTGCTGCTGGATCCGGTGATGCCGTGGCCAACGACGACGCAGCCG
CCACGGTAAGCAAAGCAACGACCCTGATCGCCGTTAATCTCTTCT
CTGCACCACCAGTTCACGTACGCCACCATTAATAATTGCCTGCCG
TAAGATAAGAAACAATTATATGGCGGTGGTGGTGCAATCATGCGA
GTACGGCGACCCGCCTTGATTTGATCCAGCTCCAGGCTCAAGGCT
CCGGCCGTATTTTTTTCCGCTCTCTTGTTTTGATTGATTGATGAG
GAGGAGAGAGAGAGCAGTAGGCGCTAGCTACTAGCTAGCTAGGGG
AAGGAGGGAGGGACGGACGTAGTAATAATTATTAAACTTTGCCAT
GTGCCTCATGTGCCCCAAAAGGTAGCAATAATTAACACTGCTGCA
CTGTTTTTTTTTAATCTGCTTCTTGTCGACTTGTCGTCGGCGGCG
ATGTCGCGGTGGACAGGCTGCTGCTGCTGCCGCCGCGTCGTCGGA
CGGCGGGCAGGAGGACGGCTGGGTGGTGTGCAGGGTGTTCAAGAA
GAAGCACCACCACAAGGAGTCAGGTGGGGGCGGGGGCAACAAGCA
CGGCAGCAGTAACAGCGAGCATGGGCACGGCGGCGCCGGCAAGGC
ATCGGCTGCGGCTGCGGCTGCGGCGCACCAGCACCAGCACCATGG
AGGCCTGCAGTACTCCTCCAGCGACGAGGCGCTGGACCAGATCCT
GCAGTACATGGGCAGGTCGTGCAAGCAGGAGCACGAGCTGGTGTC
GCCGGCGCCGGCGCCGCCGGGACGGGCGGCGGCGTCCAGGTACCT
CCGGCCCATCGAGACCGTTCTGGGCGGGCACGCGTTCATGAAGCT
TCCCGCGCTCGAGAGCCCGTCCGCGGCCGCGTCCGCATCGCTGAC
ACAGCCGGCGCAGCACGACGAGCTCTACCGCGCCGCCGGGAACGG
GATCACGGACTGGGCCATGATGGACCGGCTGGTGGCGTCGCACCT
GAACGGGCAGCAGGCGCCCGCCGCGGCGGACCAGCTCGGCGGCGG
CTGCGGCTTCGACGCGGACGCCGGCGCCGAAGACGCGGACGCCGG
CCTCGCCTTCTACTCCGCCGCCGCCAGCCGGCTGCTCGGCTCCGG
CGGCGGCGCCGGCAGCGACGACGACCTGTGGAGCTTCACGCGGTC
GTCGGTTTCGTCAACGGCGGCGGCGGCGGCCACGTCCACGGAGCG
GCTCAGCCACGTGTCACTGTAG;
zmnst2-2 mutant (SEQ ID NO: 2):
ATGAGCATCTCGGTGAACGGGCAGTCGTGCGTGCCGCCGGGGTTC
CGCTTCCACCCCACGGAGGAGGAGCTGCTCAACTACTACCTCCGC
AAGAAGGTGGCCTCCCAGGAGATCGACCTCGACGTCATCCGCGAC
GTCGACCTCAACAAGCTCGAGCCATGGGACATCCAAGGTACGTAC
GTACGCACGGCCGGTCCGGTCCGATCCGGTACCACTGCCTTCTTC
AACTTCAAGTTCAAGCACGCGCGCGCGCAGTAGCAGAGCAGCTCG
TGGTCGTGGATCAGATCGGATCGGAGCTGGTGCACGACCATGAGA
GCACGGACATGAACATGAACAGACCTTGTTGTGAATGCAACTGCC
TTAGCTAGCTACCAAGCAAGTACTCTCGTTCGTACAGCAGCATGT
ATAGCTTATGTTCGTTGATCGACAAAACTAGCTAGCAAACAATCA
AACGCGATCGAATTCATCGCGCGCTAACTACTGGCTAACAACTGC
TACTACTACTAAAGCCGCTAGCTCCATTCCATGCATGGAAATCGC
GCGCAGAGAAATGCAAGATCGGGTCGGGTCCCCAGAACGACTGGT
ACTTCTTCAGCCACAAGGACAAGAAGTACCCGACGGGGACGCGCA
CCAACCGCGCCACGGCCGCCGGGTTCTGGAAGGCCACCGGCCGCG
ACAAGGCCATCTACAACGCCGTCAAGCGCATCGGCATGCGCAAGA
CGCTCGTCTTCTACAAGGGCCGCGCGCCGCACGGCCAGAAGTCCG
ACTAGATCATGCACGAGTACCGCCTCGACGACCCCGCTGCTGCTG
CTGCTGCTGGATCCGGTGATGCCGTGGCCAACGACGACGCAGCCG
CCACGGTAAGCAAAGCAACGACCCTGATCGCCGTTAATCTCTTCT
CTGCACCACCAGTTCACGTACGCCACCATTAATAATTGCCTGCCG
TAAGATAAGAAACAATTATATGGCGGTGGTGGTGCAATCATGCGA
GTACGGCGACCCGCCTTGATTTGATCCAGCTCCAGGCTCAAGGCT
CCGGCCGTATTTTTTTCCGCTCTCTTGTTTTGATTGATTGATGAG
GAGGAGAGAGAGAGCAGTAGGCGCTAGCTACTAGCTAGCTAGGGG
AAGGAGGGAGGGACGGACGTAGTAATAATTATTAAACTTTGCCAT
GTGCCTCATGTGCCCCAAAAGGTAGCAATAATTAACACTGCTGCA
CTGTTTTTTTTTAATCTGCTTCTTGTCGACTTGTCGTCGGCGGCG
ATGTCGCGGTGGACAGGCTGCTGCTGCTGCCGCCGCGTCGTCGGA
CGGCGGGCAGGAGGACGGCTGGGTGGTGTGCAGGGTGTTCAAGAA
GAAGCACCACCACAAGGAGTCAGGTGGGGGCGGGGGCAACAAGCA
CGGCAGCAGTAACAGCGAGCATGGGCACGGCGGCGCCGGCAAGGC
ATCGGCTGCGGCTGCGGCTGCGGCGCACCAGCACCAGCACCATGG
AGGCCTGCAGTACTCCTCCAGCGACGAGGCGCTGGACCAGATCCT
GCAGTACATGGGCAGGTCGTGCAAGCAGGAGCACGAGCTGGTGTC
GCCGGCGCCGGCGCCGCCGGGACGGGCGGCGGCGTCCAGGTACCT
CCGGCCCATCGAGACCGTTCTGGGCGGGCACGCGTTCATGAAGCT
TCCCGCGCTCGAGAGCCCGTCCGCGGCCGCGTCCGCATCGCTGAC
ACAGCCGGCGCAGCACGACGAGCTCTACCGCGCCGCCGGGAACGG
GATCACGGACTGGGCCATGATGGACCGGCTGGTGGCGTCGCACCT
GAACGGGCAGCAGGCGCCCGCCGCGGCGGACCAGCTCGGCGGCGG
CTGCGGCTTCGACGCGGACGCCGGCGCCGAAGACGCGGACGCCGG
CCTCGCCTTCTACTCCGCCGCCGCCAGCCGGCTGCTCGGCTCCGG
CGGCGGCGCCGGCAGCGACGACGACCTGTGGAGCTTCACGCGGTC
GTCGGTTTCGTCAACGGCGGCGGCGGCGGCCACGTCCACGGAGCG
GCTCAGCCACGTGTCACTGTAG.

Compared with wild type B73 plant, the zmnst2-1 mutant plant and zmnst2-2 mutant plant both had the phenotype of leaf softening. The zmnst2-1 plant is crossed with the zmnst2-2 plant to obtain F1 generation (zmnst2-1/zmnst2-2) plant, then zmnst2-1/zmnst2-2 plant is tested for genetic allelism. It is found that the zmnst2-1/zmnst2-2 plant also shows leaf softening, and this indicates that mutations in the ZmNST2 gene causes the alteration in the softness of maize leaves and that zmnst2-1 and zmnst2-2 are allelic mutants of the ZmNST2 gene. There are no significant differences in other morphological aspects of the zmnst2-1 mutant plant, zmnst2-2 mutant plant, and zmnst2-1/zmnst2-2 mutant plant compared with wild type B73 plant.

### 2. Staining for Lignin of Zmnst2 Plant

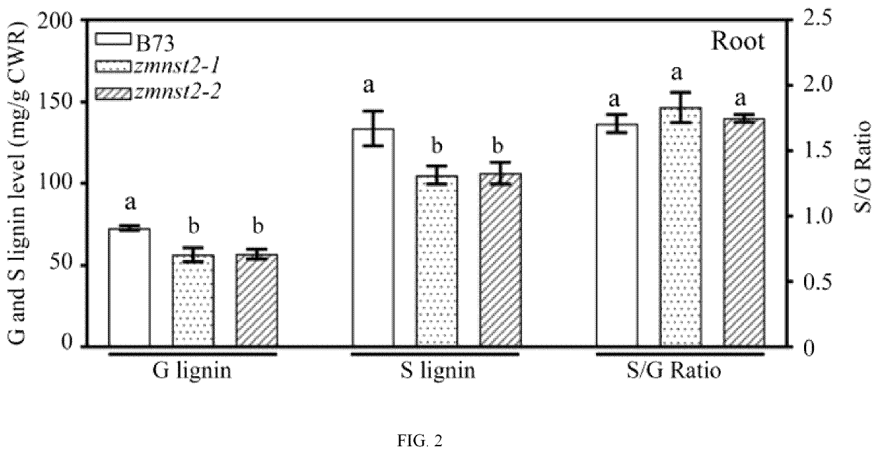

One of the reasons for the leaf softening of maize may be the reduction of lignin level. Therefore, firstly, the staining for total lignin in the roots, internodes and leaf veins of the wild type B73 plant, zmnst2-1 mutant plant, zmnst2-2 mutant plant and zmnst2-1/zmnst2-2 mutant plant was observed, as shown in FIG. 2. The results show that the staining for total lignin of the zmnst2-1 mutant plant, zmnst2-2 mutant plant, and zmnst2-1/zmnst2-2 mutant plant is significantly weaker than wild type B73 plant in the roots, internodes and leaf veins, and this indicates that the mutations of ZmNST2 result in a reduction of lignin levels in the roots, internodes, and leaf veins of the maize plants, and that the zmnst2-1 and zmnst2-2 are allelic mutants.

### 3. Analysis of Lignin Level and Cellulose Level of Zmnst2 Plant

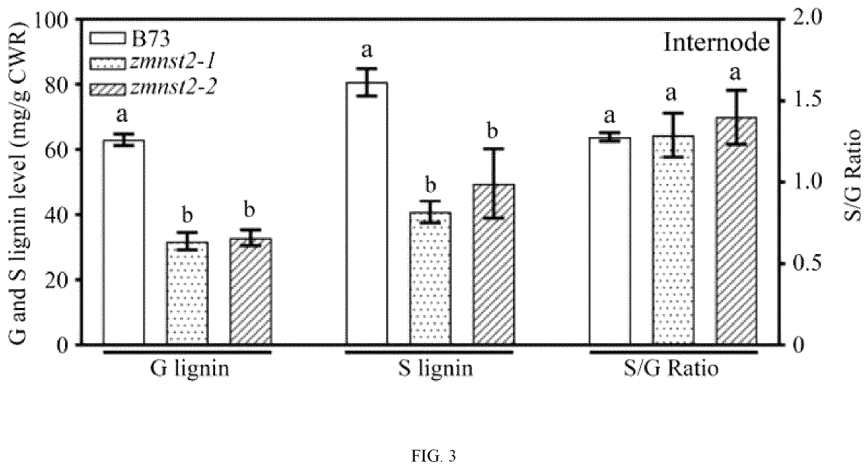

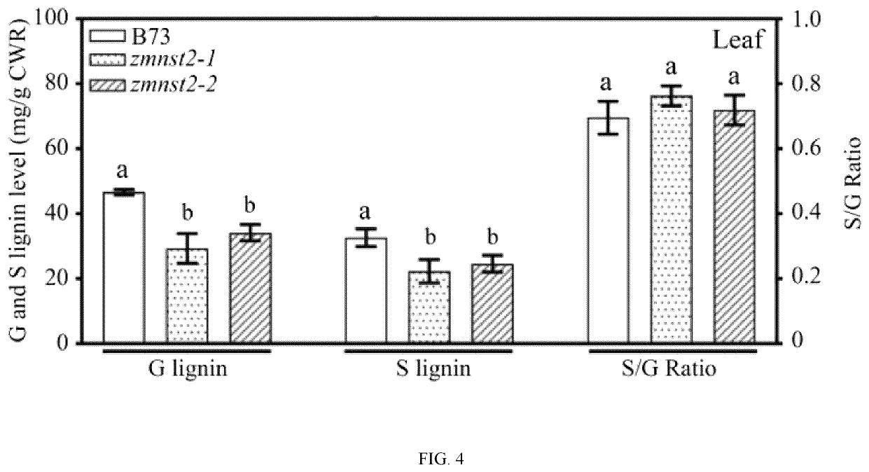

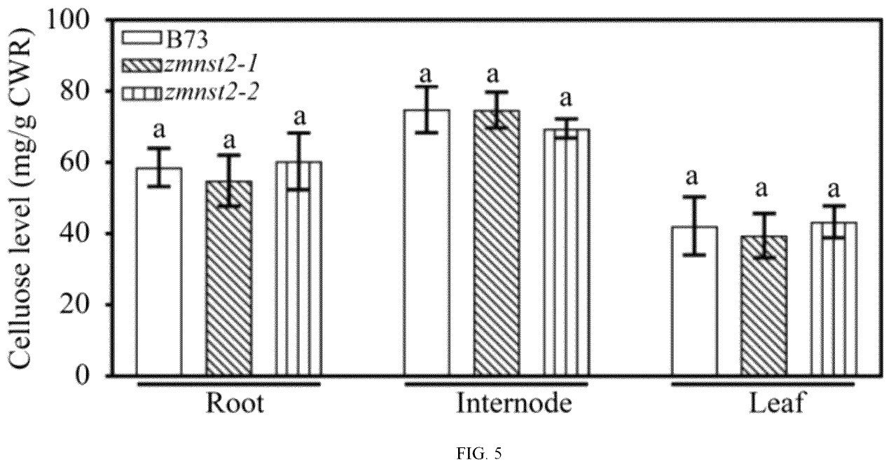

To further determine whether ZmNST2 regulates lignin accumulation, the levels of G and S lignin monomers in roots (as shown in FIG. 2), in internodes (as shown in FIG. 3), and in leaf veins (as shown in FIG. 4) of wild type B73 mutant plant, zmnst2-1 mutant plant and zmnst2-2 mutant plant are determined. The results show that the levels of G and S lignin in roots, internodes and leaf veins of both zmnst2-1 plant and zmnst2-2 plant are significantly lower than in wild type B73 plant, whereas there is no significant difference in the S/G ratio. Moreover, the cellulose levels (as shown in in FIG. 5) in roots, internodes and leaf veins of wild type B73 plant, zmnst2-1 mutant plant and zmnst2-2 mutant plant are analyzed, and the results show that there are no significant differences in the cellulose levels in roots, internodes and leaf veins of the zmnst2-1 mutant plant and zmnst2-2 mutant plant compared with that of the wild type B73 plant.

### 4. Analysis of Fermentation Indexes of Zmnst2 Plant

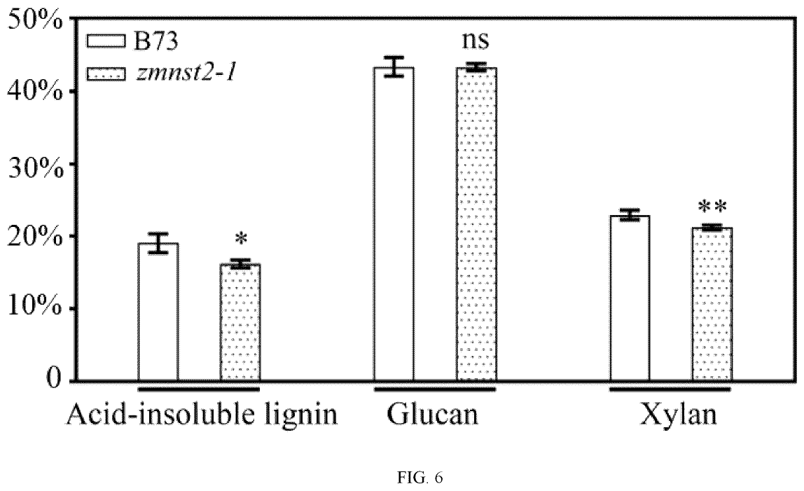

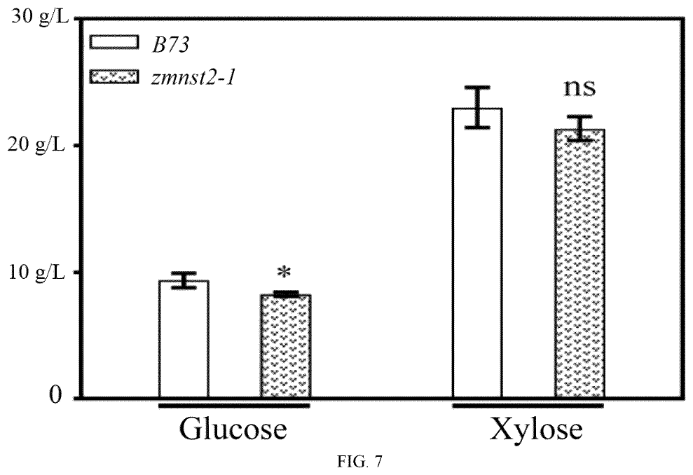

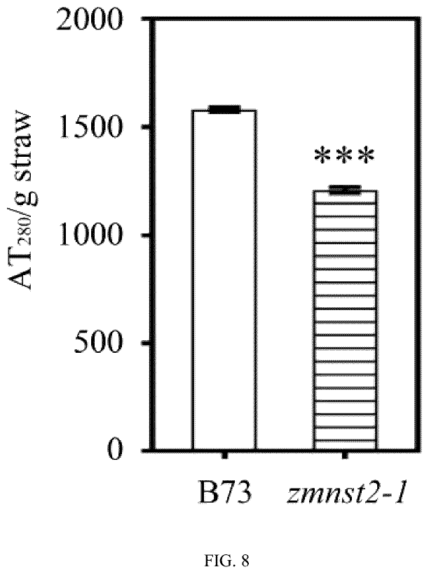

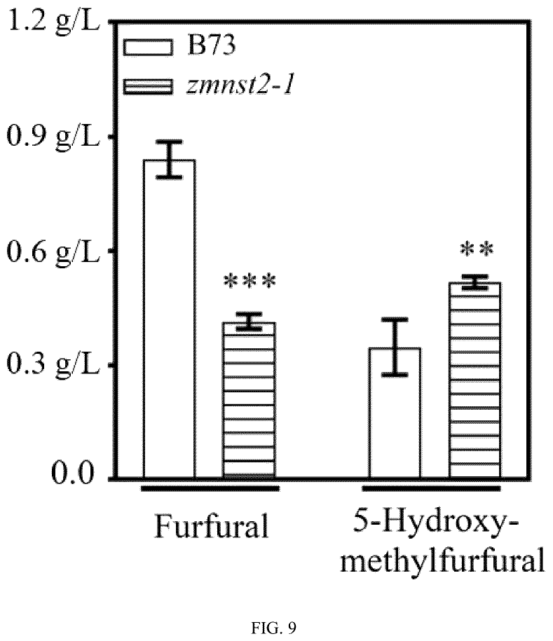

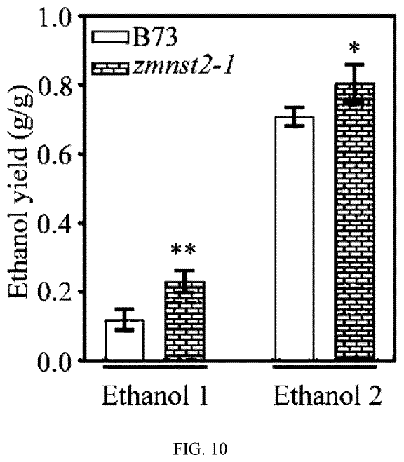

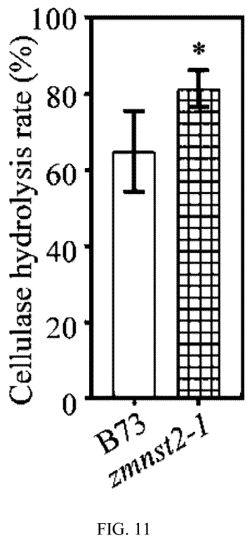

Moreover, the lignin level and cellulose level in straws of wild type B73, zmnst2-1 mutant are further determined using the NREL method, where acid-insoluble lignin represents the lignin that is insoluble in sulfuric acid, glucan represents the cellulose, and xylan represents the hemicellulose. As shown in FIG. 6, the results show that the acid-insoluble lignin level and the xylan level in zmnst2-1 mutant straw are reduced by 15.02% and 7.52%, respectively, while the level of glucan is unchanged compared with that of wild type B73, and this result is consistent with the results of the analysis of the lignin level determined by GC-MS and the cellulose level determined by anthrone colorimetry. Considering that lignin affects the binding of cellulase and cellulose and plays an important role in the production of cellulosic ethanol, and that the lignin level is significantly reduced in the zmnst2-1 mutant straw compared with the wild type B73 straw, ethanol fermentation indexes of zmnst2-1 mutant straw are determined and analyzed. As shown in FIG. 7, the results show that the fermentable material glucose level in zmnst2-1 mutant straw after pretreatment by dilute acid is reduced by 11.60% compared with wild type B73 mutant straw, whereas the xylose level is unchanged. As shown in FIG. 8 and FIG. 9, the results also show that the level of phenolic compounds (AT₂₈₀), which are inhibitory to fermentation, are all significantly reduced in zmnst2-1 mutant straw, and the level of furfural, which is toxic to fermentation, is reduced by 50.60%, whereas the level of 5-hydroxymethylfurfural is increased by 48.92%. As shown in FIG. 10, determination results of fermenting fermentation substrates to produce ethanol show that the yield of ethanol (ethanol 1) produced by co-fermentation of glucose and xylose and the yield of ethanol (ethanol 2) produced by fermentation of glucose alone after hydrolysis of cellulose by cellulase are increased by 91.89% and 13.82%, respectively, in zmnst2-1 mutant straw compared to wild type B73 mutant straw; as shown in FIG. 11, determination results of cellulase hydrolysis rate in straws of wild type B73 and zmnst2-1 mutant show that the cellulase hydrolysis rate in zmnst2-1 mutant straw is significantly increased by 25.34% compared with that wild type B73. These results show that although the mutation of ZmNST2 reduced the level of fermented material glucose by a small amount, it reduced the level of most fermentation inhibitors due to the reduction of lignin level and improved the hydrolysis of cellulose by cellulase, and further resulted in a significant increase in the efficiency of bioethanol production by fermentation.

In summary, the present disclosure provides a maize mutant with reduced lignin content and increased ethanol production from fermentation. Analysis of the results show that the mutation of ZmNST2 caused a decrease in lignin level, a decrease in fermentation inhibitors, and an increase in the hydrolysis rate of cellulase, and thereby favors the production of bioethanol by fermentation of maize straw. Therefore, this gene has a key role in the application of bioethanol production by fermentation of maize straw and is an important resource for breeding new maize varieties with high ethanol yield by fermentation.

The above-described embodiments are only descriptions of the preferred manner of the present disclosure, and are not intended to limit the scope of the present disclosure. Without departing from the spirit of the design of the present disclosure, the various deformations and improvements made by the persons of ordinary skill in the field of the technical solutions of the present disclosure shall fall within the scope of protection determined by the claims of the present disclosure.

What is claimed is:

1. A mutant gene of ZmNST2, comprising a nucleotide sequence shown in SEQ ID NO: 1 or SEQ ID NO: 2.

2. A method for creating a maize mutant, comprising introducing the mutant gene of ZmNST2 of claim 1 into a maize material to obtain the maize mutant.

3. A method for improving efficiency of bioethanol production through fermentation of maize straw, comprising the steps of the bioethanol production by fermentation of the maize mutant as claimed in claim 2.

4. The method according to claim 3, wherein the efficiency of the bioethanol production by fermentation is improved by reducing a lignin content in the maize straw.

5. The method according to claim 3, wherein the efficiency of the bioethanol production by fermentation is improved by reducing a content of a fermentation inhibitor in the maize straw.

6. The method according to claim 3, wherein the efficiency of the bioethanol production by fermentation is improved by increasing a hydrolysis rate of cellulase in the maize straw.
